(19) United States
(12) Patent Application Publication
(10) Pub. No.: US 2024/0398216 A1
(43) Pub. Date: Dec. 5, 2024

(54) ENDOSCOPY SYSTEM & ELEMENTS THEREOF

(71) Applicant: I.Q. ENDOSCOPES LTD, Chepstow, Gwent (GB)

(72) Inventors: Andrew MILLER, Chepstow, Gwent (GB); Patrick WARD-BOOTH, Chepstow, Gwent (GB); Daniel Peterson GODFREY, Cambridge (GB)

(21) Appl. No.: 18/691,582

(22) PCT Filed: Sep. 6, 2022
(86) PCT No.: PCT/EP2022/074719

§ 371 Date: Mar. 13, 2024

(30) Foreign Application Priority Data
Sep. 14, 2021 (GB) 2113121.4

(51) Int. Cl. A61B 1/005

(52) U.S. Cl. CPC A61B 1/0052; A61B 1/0057

(57) ABSTRACT

Various embodiments provide an imaging endoscopy system comprising a hand controller, an insertion section having a proximal end connected to the hand controller and a distal end for insertion into a subject, the distal end having a steering section and a distal tip, and a steering control mechanism comprising a first continuous steering wire. The first continuous steering wire has first and second ends coupled to a user control assembly at the hand controller, and an intermediate portion fixed at the distal tip of the insertion section, thereby defining two steering wire portions extending from the hand controller to the distal tip, said steering wire portions capable of being tensioned to cause bending of the steering section in response to operation of the user control assembly of the hand controller.

This application claims priority from GB2113121.4 filed 14 Sep. 2021, the contents and elements of which are herein incorporated by reference for all purposes.

### FIELD OF THE INVENTION

The present invention relates to imaging endoscope systems, elements thereof and particularly to a hand controller, steering system and components thereof for use in an imaging endoscope systems.

### BACKGROUND

It is known that early detection of disease may be assisted by endoscopic examination of internal structures such as the alimentary canals and airways, e. g., the oesophagus, lungs, colon, uterus, and other organ systems. Endoscopic examination may be carried out using an imaging endoscope. Typically, an imaging endoscope has a flexible tube providing a light guide that guides illumination light from an external light source located proximally of the flexible tube to a distal tip of the flexible tube. The illumination light thereby illuminates the tissue to be examined. The distal tip typically also includes an objective lens to gather light from the tissue being examined. The flexible tube provides an imaging light guide to carry the light away from the distal tip and to a camera at the proximal end of the flexible tube. It is also known for the imaging light guide to be dispensed with and replaced with an imaging camera chip at the distal tip. In this case, the signal from the imaging camera chip is conducted along electrical wires in the flexible tube. Based on either approach, an image is produced for display to the imaging endoscope operator.

It is also known, in addition to the imaging function described above, for imaging endoscopes to provide further functionality. For example, it is known for the distal tip to be equipped with a port for dispensing air (or more generally, insufflation gas) to inflate the internal structure being examined, a port for dispensing irrigation liquid such as water or saline, and a port for a medical tool such as biopsy forceps. To allow such services to be provided, the flexible tube typically provides suitable lumens dedicated to the provision of these services.

Although some endoscopic procedures can be carried out with a rigid insertion tube, for many procedures it is preferred that the insertion tube is flexible. This allows the insertion tube to be located along tortuous and often complex paths within the body. To allow adequate steering of the flexible tube within the body, it is known to provide the distal tip of the insertion tube with means for deflecting the distal tip, and therefore with means to steer the distal tip. A typical approach to this is to provide control wires, extending along the flexible tube from a hand controller at a proximal end of the flexible tube and anchored to the distal tip. The hand controller has control knobs to allow movement of the control wires and consequently steering of the distal tip.

One example of a conventional hand controller and steering system is described in an earlier application by the present inventors, GB2569013 B—Imaging endoscope system and associated methods, published 9 Sep. 2020, which is herein incorporated by reference.

However, the present inventors have realised that there are a number of opportunities for improvement of existing hand controller architecture, in particular in relation to the steering mechanism incorporated therein, and components thereof.

### SUMMARY OF THE INVENTION

The present inventors have realised a number of innovations which provide improvements on existing hand controller or steering mechanism architecture within an endoscope. These various innovations may reduce the overall cost and complexity of manufacture of the hand controller or steering control mechanism, whilst providing suitable or improved performance in comparison to conventional endoscopes.

These innovations are referred to herein as ‘Innovation A’, ‘Innovation B’, ‘Innovation C’, and ‘Innovation D’.

Whilst discussed separately, it will be understood that these innovations are nevertheless combinable, and accordingly, the invention includes any combination of the innovations, aspects of innovations and preferred features described except where such a combination is clearly impermissible or expressly avoided.

### Innovation A

In a first aspect of Innovation A, the present invention provides a steering control mechanism configured for controlling bending of a steering section of an endoscope in a first plane via first and second steering wires, the steering control mechanism comprising:
a first rotational drive wheel comprising first and second drive wheel body portions, the first drive wheel body portion comprising a first drum element coupled to the first steering wire, and the second drive wheel body portion comprising a second drum element coupled to the second steering wire,
wherein the rotational drive wheel is configured to provide one-way relative rotation of the first drum element and the second drum element.

The term ‘rotational drive wheel’ is used herein to refer to a wheel which drives steering movement of the control wires by rotation of the wheel. As discussed below in relation to optional features of the present invention, this drive wheel may itself be driven by a control mechanism, directly or via a gearing arrangement.

Providing an arrangement in which the rotational drive wheel is configured to provide one-way relative rotation of the first drum element and the second drum element can allow the steering control mechanism to be adjusted e.g. during manufacture of the steering control mechanism, for example to adjust the tension in the first and second steering wires in a more facile manner than is possible in known systems. Specifically, as the first drum element and the second drum element can be rotated relative to one another in only one rotational direction, it is possible to adjust the relative position, effective lengths and/or the tension of the first and second steering wires which are respectively coupled to the first and second drum elements, via this one-way relative rotation.

Accordingly, in a second aspect of Innovation A, the present invention therefore provides a method of assembling a steering control mechanism, the steering control mechanism comprising:
a first rotational drive wheel comprising first and second drive wheel body portions, the first drive wheel body portion comprising a first drum element coupled to the first steering wire, and the second drive wheel body portion comprising a second drum element coupled to the second steering wire,
wherein the rotational drive wheel is configured to provide one-way relative rotation of the first drum element and the second drum element, and wherein the method includes at least one step of rotating the first drum element relative to the second drum element.

The step of rotating the first drum element relative to the second drum element may be performed manually by a human operator. Alternatively, it may be performed by an assembly machine. Rotating the first drum element relative to the second drum element may adjust the relative position or the effective lengths of the first and second steering wires. For example, in one preferred arrangement, the first and second drum elements may be rotatable relative to one another to wind the first steering wire onto or off the first drum element, and/or the second steering wire onto or off the second drum element. Winding a steering wire onto the drum element will decrease its effective length and/or increase the tension in the steering wire. Winding a steering wire off a drum element will increase its effective length and/or decrease the tension in the steering wire.

The step of rotating the first drum element relative to the second drum element may be performed to achieve or set a predetermined tension in one or both of the wires forming part of the steering control mechanism. In other words, the steering wires may be pre-tensioned during the manufacturing process. For example, the tension in each wire forming part of the steering control mechanism may be set to an amount of 1 N or more, up 20 N or less. In some cases, the tension may be set at an amount of 2 N or more, 3 N or more, 4 N or more or 5 N or more. In some cases, the tension may be set at an amount of 20 N, 15 N or less, or 10 N or less. It may be preferred that the maximum load on the wires is 10 N or less, as if the tension in the system is too high, there may be increased risk of damage to the steering wire during use of the system or over time (e.g. during storage).

Further optional features of the steering control mechanism will now be discussed.

The first drum element and the second drum element may be fixed relative to, or integrally formed with, the first and second drive wheel body portions. In such arrangements, the rotational drive wheel will be configured to provide one-way relative rotation of the first and second drive wheel body portions as a whole. Where the drum elements are fixed relative to, or integrally formed with, the first and second drive wheel body portions, providing for one-way relative rotation of the first and second drive wheel body portions as a whole will inherently also provide for one-way relative rotation of the first and second drum elements. In preferred arrangements, the first drum element and the second drum element are integral parts of the first and second drive wheel body portions. Integrally forming these parts can allow the drive wheel body portions to each be produced in a single moulding step, thereby allowing for low manufacturing costs. Alternatively, the first and second drum elements may be discrete elements forming part of the first and second drive wheel body portions (but, e.g. rotatable relative to the first and second drive wheel body portions).

The way in which one-way rotation of the first and second drum elements is provided is not particularly limited, however in one suitable arrangement, the rotational drive wheel may comprise a ratchet mechanism. The ratchet mechanism may include at least one toothed gear surface, and at least one pawl. For example, one of the first drive wheel body portion and the second drive wheel body portion may comprise the at least one toothed gear surface, and the other of the first drive wheel body portion and the second drive wheel body portion may comprise the at least one pawl, with the first and second drive wheel body portions being configured to provide for interaction of the toothed gear surface and the pawl to thereby allow for one-way relative rotation of the first and second drum elements. A rachet arrangement is a convenient and relatively cheap way to implement one-way relative rotation of two components.

The location of the toothed gear surface and the pawl on the first and second drive wheel body portions is not particularly limited. They may be provided on the drum elements, or on another part of the first and second drive wheel body portions. For example, the toothed gear surface may be an internal toothed gear surface formed on an inner surface of a drive wheel body portion, with the pawl arranged to engage with said internal toothed gear surface. Alternatively, the toothed gear surface may be an external toothed gear surface formed on an external surface of a drive wheel body portion, with the pawl arranged to engage with said external toothed gear surface. The toothed gear surface may be formed by any suitable manufacturing method: as one example, it may be formed during moulding of the drive wheel body portion. As a further example, it may be formed by mechanically shaping the drive wheel body portion (by cutting or similar) after the initial moulding process.

The pawl may be provided as pivoted element (i.e. pivotable about a pivot point) with the free end of the pivotable element being arranged to engage with the teeth of the toothed gear surface. Alternatively the pawl may be provided as part of a flexible member. In one convenient arrangement, the pawl is provided as part of a flexible arm, with the free end of the flexible arm being arranged to engage with the teeth of the toothed gear surface. Where the pawl is provided as part of a flexible arm, the flexible arm is preferably integrally formed with the respective drive wheel body portion. Integrally forming such arms can allow the drive wheel body portion to be produced in a single moulding step, thereby allowing for low manufacturing costs. Furthermore, arms formed integrally with their respective drive wheel body portion may be more durable than those formed separately and attached in a subsequent manufacturing step.

In some arrangements, more than one pawl may be provided for engagement with one or more respective toothed gear surfaces. For example, two or more, three or more or more or more pawls may be provided. Where multiple pawls are provided, these may be equiangularly spaced about the drive wheel body portion which they are part of. The multiple pawls may be arranged to engage with the same toothed gear surface, just at different locations. Alternatively, the multiple pawls may be configured to engage with different toothed gear surfaces.

In one preferred arrangement, the first drive wheel body portion comprises a single continuous toothed gear surface which is formed as an internal toothed gear surface, and the second drive wheel body portion comprises a plurality of pawls equiangularly spaced around the second drive wheel body portion, each pawl being provided as part of a flexible arm integrally formed with the second drive wheel body portion, wherein the free end of each flexible arm is arranged to engage with the teeth of the continuous toothed gear surface of the first drive wheel body portion.

The first and second drive wheel body portions may be formed in any suitable manner. Conveniently, they may be injection moulded parts.

The first and second drive wheel body portions may be formed from a polymeric material. For example, they may be formed from a material selected from the group consisting of polyesters, polyamides (e.g. nylons), acetyls, polycarbonates or any other suitable polymer. Polymeric materials are generally cheap, are relatively facile to shape and/or mould during manufacture.

The first and second drum elements may be coupled to their respective steering wire by a portion of the respective steering wire being fixed relative to the respective drum element. In preferred arrangements, an end of the steering wire is fixed relative to its respective drum element. Fixing of at least a portion of the steering wire relative to the drum element may be achieved by provision of a mechanical fixing—for example, via clamping or gripping means such as a ferrule, by tying the steering wire to the drum element, or by any other suitable mechanical fixing means. Alternatively or additionally, this fixing may be provided by a chemical bond (e.g. by an adhesive)

In one convenient arrangement, the first drum element and the second drum element each comprise respective wire-receiving recesses in which ends of the first steering wire and the second steering wire are respectively affixed. However, other arrangements are envisaged. For example, the steering wires may simply be attached to an outer surface of the drum element. Alternatively, they may be attached to a projection formed on the drum element. Alternatively, they may be attached to another part of the drive wheel body portion and coupled to the drum element by at least a part of the steering wire being wrapped around at least a portion of the drum element.

In a preferred arrangement, the first and second drum elements each comprise respective wire-receiving recesses having a ferrule disposed inside and configured to receive the end of a steering wire, the ferrule being clamped during manufacture to affix the end of the steering wire in the wire-receiving recess.

In a conventional endoscope, it is generally advantageous to be able to steer a steering section of the endoscope in both a first and second plane, to allow for movement of the steering section in both of these planes. Accordingly, in a preferred arrangement, the steering control mechanism is further configured to control bending of a steering section of an endoscope in a second plane via third and fourth steering wires, the steering control mechanism comprising:
a second rotational drive wheel comprising respective first and second drive wheel body portions, the first drive wheel body portion comprising a first drum element coupled to the third steering wire, and the second drive wheel body portion comprising a second drum element coupled to the fourth steering wire.

Preferably, the second rotational drive wheel is configured to provide one-way relative rotation of its respective first drum element and second drum element. This can allow the third and fourth steering wires to be adjusted by the one-way relative rotation of the first and second drum element in the same manner as discussed above in relation to the first and second steering wires.

Where the steering control mechanism comprises first and second rotational drive wheels, these may be arranged to be substantially adjacent to one another. The first and second rotational drive wheels may share a common axis of rotation. This can help to ensure that the steering mechanism is relatively compact.

A washer may be interposed between the first and second rotational drive wheel. This can help to avoid entanglement of the steering wires coupled to the first and second rotational drive wheel respectively.

One or more user input control elements may be provided to control rotation of the rotational drive wheel(s) based on user input to the user input control elements. For example, the steering control mechanism may comprise one or more rotatable control wheels, switches, levers or buttons which may be configured to control rotation of the rotational drive wheel(s) based on user input to these elements—e.g. by transmission of torque to the rotational drive wheel(s). An assembly including such user input control elements in the steering control mechanism may be referred to as a user control assembly.

In preferred arrangements, the steering control mechanism may comprise a first rotatable control wheel configured to transmit torque to the first rotational drive wheel. The steering control mechanism may comprise a second rotatable control wheel configured to transmit torque to the second rotational drive wheel (where present). In a preferred arrangement, the steering control mechanism comprises two rotatable control wheels, the first rotatable control wheel being configured to transmit torque to the first rotational drive wheel to drive bending of the steering section of the endoscope in the first plane, and the second rotatable control wheel being configured to transmit torque to the second rotational drive wheel to drive bending of the steering section of the endoscope in the second plane.

Rotation of the rotatable control wheels may transmit torque to the rotational drive wheels directly or indirectly. The first and second rotatable control wheels may be arranged to transmit torque to the first and second rotational drive wheels via respective first and second drive shafts. Such first and second drive shafts may be concentrically arranged. This can help to ensure that the steering mechanism is relatively compact.

In a direct-drive arrangement, the first rotational drive wheel may be arranged in a keyed configuration with respect to a first drive shaft coupled to a first rotatable control wheel. The second rotational drive wheel may be arranged in a keyed configuration with respect to a second drive shaft coupled to a second rotatable control wheel. In this way, torque resulting from a user rotating the rotatable control wheels can be transmitted directly to the rotational drive wheels via the drive shafts.

In an indirect-drive arrangement, the first rotational drive wheel may be coupled to a first rotatable control wheel via a gearing arrangement. The second rotational drive wheel may be coupled to a second rotatable control wheel via a similar gearing arrangement. The gearing arrangement may include e.g. a first gear arranged in a keyed configuration with respect to a drive shaft coupled to a rotatable control wheel, said first gear being configured to translate rotational movement of the first rotatable control wheel to the rotational drive wheel, which may constitute a second gear. An indirect-drive arrangement may have some advantages over a direct drive arrangement, as it is possible to change the gearing ratio of the steering control mechanism by selection of an appropriate gearing arrangement.

As mentioned above, the steering control mechanism discussed above is configured for controlling bending of a steering section of an endoscope.

Accordingly, in a third aspect of Innovation A, the present invention provides an imaging endoscope, or an imaging endoscopy system, comprising:
a base unit;
a hand controller including the steering control mechanism of the first aspect;
an umbilical section for releasably connecting the base unit and the hand controller;
an insertion section having a proximal end connected to the hand controller and a distal end for insertion into a subject, the distal end having a steering section and a distal tip, wherein bending of the steering section is controllable by user input to the steering control mechanism.

Further optional features of the imaging endoscope/imaging endoscopy system will now be discussed.

The hand controller may be configured to be releasably connectable to a base unit by means of an umbilical section. The base unit may provide electrical power, irrigation liquid and insufflation gas to the insertion section, via the umbilical section and hand controller.

The distal tip assembly may include a light source for illumination of a region of tissue of interest. Preferably, the light source includes at least one light emitting diode (LED). There may be provided at least one electrical conductor along the insertion section for providing electrical power to the light source.

The distal tip assembly may further include an imaging chip for imaging the region of tissue of interest. There may be provided at least one electrical conductor along the insertion section for providing electrical power to the imaging chip. There may be provided at least one electrical conductor for conducting electrical signals from the imaging chip to the proximal end of the insertion section. In this manner, it is possible to implement an imaging endoscopy system in which there is no need for the insertion section to transmit light, whether as illumination or optical images.

The distal tip may include a distal tip housing comprising light-transmissive portion. The distal tip housing may be integrally formed from a polymeric material.

The distal tip housing may have a collar portion extending proximally of a distal end face. The collar portion may be adapted to fit over a distal end of the steering section. This is considered to be a convenient implementation.

The distal tip housing may have a cleaning nozzle arranged at the distal end face, to direct irrigation liquid to clean a lens of the imaging chip.

The steering wires of the steering control mechanism may extend along the insertion section and be fixed at or near the distal end of the insertion section. In this way, the steering section of the insertion section may be bendable for steering by application of tension to the steering wires by operation of the hand controller. There may be provided such four steering wires, the four steering wires being substantially equiangularly spaced around the insertion section when viewed in cross section—these steering wires corresponding to the first to fourth steering wires discussed above.

The steering wires may have sheaths each defining an axis of constrained movement for each respective steering wire. In other words, preferably the steering wires are provided as Bowden cables. Preferably, the steering wires extend through the steering section to the distal tip and the sheaths of the steering wires do not extend through the steering section. In this way, application of tension to the steering wires can cause bending primarily in the steering section of the insertion section, without substantially influencing the curvature of the remaining portion of the insertion section intermediate the hand controller and the steering section.

Other features of the imaging endoscope, or imaging endoscopy system may be implemented as described in GB2569013, referenced above.

### Innovation B

Steerable endoscopes are typically not customisable in terms of their steering response (i.e. steering output based on a set user input). However, the present inventors have realised that it may be advantageous to offer a steering control mechanism which is adjustable (either during the manufacturing process, or subsequently by a user) in order to adjust the steering response of the steering control mechanism.

According, in a first aspect of Innovation B, the present invention provides a kit of parts comprising:
a steering control mechanism configured for controlling bending of a steering section of an endoscope in a first plane via first and second steering wires; and
one or more spacing elements;
wherein the steering control mechanism comprises a first rotational drive wheel comprising first and second drive wheel body portions, the first drive wheel body portion comprising a first drum element coupled to the first steering wire, and the second drive wheel body portion comprising a second drum element coupled to the second steering wire; and
wherein the one or more spacing elements are configured to be disposed on the first and/or second drum elements to thereby change the apparent diameter of the first and/or second drum elements, thereby changing the steering response of the steering control mechanism.

The kit will typically be provided as part of an endoscopy system. In preferred arrangements, the endoscopy system will be supplied with selected spacing elements pre-fitted (i.e. fitted during the manufacturing process, for example in response to a customer order for an endoscopy system have a selected steering response).

Accordingly, in a second aspect of Innovation B, the present invention provides an imaging endoscopy system, comprising:
a base unit;
an umbilical section for releasably connecting the base unit and the hand controller;
an insertion section having a proximal end connected to the hand controller and a distal end for insertion into a subject, the distal end having a steering section and a distal tip; and
a hand controller including a steering control mechanism configured for controlling bending of a steering section of the endoscope in a first plane via first and second steering wires, wherein the steering control mechanism comprises a first rotational drive wheel comprising first and second drive wheel body portions, the first drive wheel body portion comprising a first drum element coupled to the first steering wire, and the second drive wheel body portion comprising a second drum element coupled to the second steering wire such that bending of the steering section is controllable by user input to the steering control mechanism,
and one or more spacing elements which are configured to be disposed on the first and/or second drum elements to thereby change the apparent diameter of the first and/or second drum elements, thereby changing the steering response of the steering control mechanism.

The term ‘apparent diameter’ is used herein to describe the composite diameter of the drum elements including the additional spacing element. By provided an arrangement in which the apparent diameter of the drum elements is configurable in this manner, it is possible to adjust the steering response of the steering control mechanism. For example by providing a larger apparent diameter of the drum elements, a set user input may result in a larger steering response (i.e. greater deflection of the steering section of the endoscope for the given user input). By providing a smaller apparent diameter of the drum elements, a set user input may result in a smaller steering response (i.e. smaller deflection of the steering section of the endoscope for the given user input).

Furthermore, the effective loading on the steering control mechanism required to steer the wires may also depend on the apparent diameter of the drum elements. For example by providing a larger apparent diameter of the drum elements, a larger input force (for example, in the case of a steering mechanism including a user input control element such as a rotatable control wheel, a large rotational force applied to the control wheel) may be required to provide steering response. Conversely, by providing a smaller apparent diameter of the drum element, a smaller input force may be required to provide a steering response.

In this way, the kit or endoscopy system may have greater utility for use by multiple different users. This is because different users may have different personal preference as to the ‘ideal’ steering response of the endoscope. For example, some users may prefer a larger steering response for a given user input—this may allow for large deflections to be performed relatively quickly. Conversely, some users may prefer a smaller steering response for a given user input, which may allow for fine motor control of the steering section. Additionally, for systems including manual user input control elements such as a rotatable control wheel, some users may prefer the feel of a ‘heavier’ or ‘lighter’ loading on the rotatable control wheel during steering. The kit or endoscopy system may comprise a plurality of spacing elements configured to change the apparent diameter of the first and/or second drum elements by different amounts. For example, the kit may comprise a plurality of spacing elements of different diameters. For example, it may include spacing elements having two or more, three or more, or four or more different outer diameters. This can allow for greatest customizability.

The spacing elements may be e.g. washers, or ring-shaped members, or they may have any other appropriate form, provided that they can be disposed on the first and/or second drum elements to thereby change the apparent diameter of the first and/or second drum elements. Where appropriate, the spacing elements may have one or more cut-out portions e.g. to allow passage of a steering wire through the spacing element.

The material and precise form of the spacing element(s) is not particularly limited, and may be formed from e.g. a polymeric material, a metal, ceramic, or any other suitable material. In preferred arrangements, the spacing element(s) are formed from a polymeric material. Polymeric materials may be preferred due to their lower density and cost as compared with other available materials. For example, they may be formed from a material selected from the group consisting of polyesters, polyamides (e.g. nylons), acetyls, polycarbonates or any other suitable polymer.

The spacing element(s) may be integrally moulded parts. In other words, they a single component formed in one piece. They may be formed by e.g. injection moulding, casting, or any other suitable manufacturing process.

The spacing element(s) may be held in place by a friction-fit arrangement, or by a fastening arrangement such as one or more clips, or via an adhesive.

In preferred arrangements, in use, at least two spacing elements are provided for each rotational drive wheel forming part of the steering mechanism, in other words, the spacing elements may be provided in pairs. For example, for a single rotational drive wheel arrangement, preferably a first spacing element is provided to be disposed on the first drum element, and a second spacing element to be disposed on the second drum element, to thereby change the apparent diameter of both of the first and second drum elements. Preferably these first and second spacing elements change the apparent diameter of both of the first and second drum elements by the same amount: in other words, these first and second spacing elements forming a pair of spacing elements preferably have the same outer diameter. For a two-rotational drive wheel arrangement, at least four spacing elements may be provided (two pairs), to be disposed on the first and second drum elements of each of the first and second rotational drive wheels. This arrangement allows for even adjustment of the steering response in all directions.

The steering control mechanism incorporated in the kit or the imaging endoscopy system may be a steering control mechanism as set out above in relation to Innovation A, or it may be a conventional steering control mechanism. The steering wire arrangement may be as set out below in relation to Innovation C, or it may be a conventional steering wire arrangement.

### Innovation C

As described above in relation to innovation A, an imaging endoscope or imaging endoscopy system will typically include four steering wires, including a first pair of steering wires arranged to control bending of a steering section of the endoscope in a first plane, and a second pair of steering wires arranged to control bending of a steering section of the endoscope in a second plane.

In conventional arrangements, these four steering wires are commonly provided as four separate components, with a first end of each of the four separate steering wires being fixed at the steering section of the endoscope, and with a second end of each of the four separate steering wires being coupled to a user control assembly at the hand controller of the endoscope.

In other known arrangements, for example as described in GB2569013 B, each of the first pair and second pair of steering wires may be formed of a continuous wire which extends from the steering section to wrap at least partially around a first rotational drive wheel (part of the steering control mechanism) and then extends back to the steering section—in other words, the first and second steering wires are part of the same continuous steering wire having two ends, with each end of the steering wire being fixed at the steering section, and an intermediate portion of the wire being wrapped around the rotational drive wheel.

However, these known arrangements have some disadvantages. In particular, the use of four separate steering wires can increase the complexity of manufacture due to the need to carefully align the relative lengths of the separate pieces of wire. Furthermore, the use of continuous steering wires which are arranged with each end of the steering wire being fixed at the steering section, and an intermediate portion of the wire being wrapped around the rotational drive wheel may not be appropriate for all steering control mechanism arrangements.

Accordingly, in a first aspect of Innovation C, the present invention provides an imaging endoscope or imaging endoscopy system comprising:
a hand controller;
an insertion section having a proximal end connected to the hand controller and a distal end for insertion into a subject, the distal end having a steering section and a distal tip;
a first continuous steering wire, said first continuous steering wire having first and second ends coupled to a user control assembly at the hand controller, and an intermediate portion fixed at the distal tip of the insertion section, thereby defining two steering wire portions extending from the hand controller to the distal tip, said steering wire portions capable of being tensioned to cause bending of the steering section in response to operation of the user control assembly of the hand controller.

In effect, this arrangement is the opposite to that disclosed in GB2569013 B, in that an intermediate portion of the steering wire is fixed at the steering section, and the respective ends of the wire are coupled to a user control assembly at the hand controller.

Such an arrangement may improve simplicity of the design and ease of manufacture of the system, in particular by removing the need to carefully align the relative lengths of the separate pieces of wire in comparison to systems using two separate steering wires, and also by offering a convenient solution for certain steering control mechanisms in which arrangements such as that disclosed in GB2569013 B are not easily implemented.

The imaging endoscope or imaging endoscopy system may further comprise a second continuous steering wire, said second continuous steering wire having first and second ends coupled to the user control assembly at the hand controller, and an intermediate portion fixed at the distal tip of the insertion section, thereby defining two further steering wire portions extending from the hand controller to the distal tip, said steering wire portions capable of being tensioned to cause bending of the steering section in response to operation of the user control assembly of the hand controller. In such an arrangement there will be a total of four steering wire portions. The four steering wire portions may conveniently be substantially equiangularly spaced around the insertion section when viewed in cross section.

These four steering wire portions may be conceptually divided into two pairs of opposed steering wire portions, wherein a first pair of steering wire portions is configured to cooperate to steer the distal tip in a first predetermined plane, and wherein a second pair of steering wire portions is configured to cooperate to steer the distal tip in a second predetermined plane. The opposed steering wire portions may be arranged at 180° about the circumference of the steering section.

In some arrangements, each pair of opposed steering wire portions are part of the same continuous steering wire.

In other arrangements, each pair of opposed steering wire portions includes one portion constituting part of the first continuous steering wire, and one portion constituting part of the second continuous steering wire. Such an arrangement may be advantageous, as the intermediate portion on the continuous steering wire which is fixed at the distal tip of the insertion section may not be required to extend across the distal tip to allow for the pair of steering wire portions to be opposed (i.e. arranged at 180° about the circumference of the steering section) to one another. Rather, the steering wire portions can be adjacent steering wire portions, arranged at less than 180° relative to one another—e.g. arranged at 90° relative to one another.

As set out above, each continuous steering wire has first and second ends coupled to a user control assembly at the hand controller. The term ‘user control assembly’ is used herein to define an assembly (such as a steering control mechanism) including one or more user input control elements (for example one or more rotatable control wheels, switches, levers or buttons) configured to control bending of the steering section by suitable tensioning of the steering wire portions. The user control assembly may comprise one or more rotational drive wheels which drive steering movement of the control wires by rotation of the wheel. Such rotational drive wheel may be themselves driven directly or indirectly (for example via a gearing arrangement) by the user control elements. The user control assembly may incorporate a steering control mechanism as described above in relation to Innovation A or Innovation B, or it may alternatively incorporate a conventional steering control mechanism.

The manner of fixing of the intermediate portion of the first and/or second continuous steering wires at the distal tip is not particularly limited: any suitable fixing may be appropriate. In one example of a suitable fixing arrangement, the intermediate portion may be fixed at the distal tip via a clamping member, such as a ferrule. In another example, the intermediate portion of the wire may be fixed using a knot—e.g. a mid loop knot such as a ‘FIG. 8’ knot. The intermediate portion of the wire may be threaded through a hole formed in the distal tip of the steering section before the knot is tied, with the knot then acting to prevent the wire being pulled through said hole. In other words, the wire may be threaded through a hole formed in the distal tip of the steering section and fixed via a knot arranged distally of said hole.

In yet another example, the intermediate portion of the wire may be fixed using an adhesive. Physical fixing arrangements such as knotting or use of a clamping member may be preferred over use of an adhesive, as there is lower risk of the wire becoming unattached from the distal tip during use. Fixing the wire using a knot may be preferred over other physical clamping arrangements such as use of a ferrule to clamp the intermediate portion of the wire, as this can lower cost of manufacture by reducing the total number of components required.

Further optional features of the imaging endoscope/imaging endoscopy system described above in relation to Innovations A and B and below in relation to Innovation D are also applicable to the imaging endoscope/imaging endoscopy system of Innovation C.

### Innovation D

In addition to the above innovations relating to design and construction of various components of the steering control mechanism for an imaging endoscope, the present inventors have realised that there is potential for further innovation in respect of allowing for greater customizability of hand controllers for use in endoscopes in general.

One typical construction for a hand controller of an imaging endoscope, as described in GB2569013 B, is for the hand controller to be formed of two main injection moulded parts which attach to each other along a longitudinal join, with steering wire controls being provided externally on the hand controller, whereby turning of the steering wire controls is transmitted into tension in the steering wires in order to control the bending of the steering section. The shape of the injection moulded parts is designed to be generally comfortable for a wide range of users. However, this standard injection moulded arrangement does not take into consideration the fact that endoscopes may be used by a wide variety of different users, with different hand sizes, or personal preference for how a hand controller should fit in the hand during use—i.e. the ‘hand-feel’ of the hand controller.

Accordingly, in a first aspect of Innovation D, the present invention provides a hand controller for an endoscopy system, the hand controller comprising a chassis sub-assembly including one or more frame portions configured to support a steering control mechanism of the hand controller; and
at least one removably attachable casing component, the removably attachable casing component being configured to be removably attachable to one or more of (i) the chassis sub-assembly; or (ii) another casing component, to thereby provide at least part of an outer shell of the hand controller.

In a second aspect of Innovation D, the present invention provides a kit of parts comprising:
a hand controller for an endoscopy system, the hand controller comprising a chassis sub-assembly including one or more frame portions configured to support a steering control mechanism of the hand controller; and
two or more interchangeable and removably attachable casing components, the interchangeable removably attachable casing components being configured to be removably attachable to one or more of (i) the chassis sub-assembly; or (ii) another casing component, to thereby provide at least part of an outer shell of the hand controller, wherein the shape of the two or more interchangeable removably attachable casing components differs.

By providing a kit in which multiple interchangeable casing components of different shapes are provided, a user of the hand controller is able to select an appropriate casing component prior to use of the hand controller, to customise the shape of the outer shell of the hand controller to meet their personal preference for controller ‘hand feel’, or to better suit the size of their hand.

The shape of the interchangeable casing components may differ in terms of size. For example, one of the casing components may be smaller than the other of the casing components. In some cases the kit may include ‘small’, ‘medium’ and ‘large’ casing components to better suit different user hand sizes.

Alternatively or additionally, the shape of the interchangeable casing components may differ in terms of their contours. For example, one of the casing components may have a flatter profile, and another of the interchangeable casing components may have a more rounded profile. The profile of the casing component can be indicated by e.g. a radius of curvature of at least a portion of the casing component.

The number of interchangeable casing components is not particularly limited. In some arrangements, three or more, four or more, or five or more interchangeable casing components may be provided as part of the kit. This can allow the kit to cater for use by a large number of potential users with different preferences for controller hand feel.

The manner in which the interchangeable casing components attach to the chassis sub-assembly or to another casing component is not particularly limited. One or more clips or fasteners may be provided on the chassis sub assembly or casing components, arranged to allow the interchangeable casing components to be removably attachable to the chassis sub assembly or casing component. In one preferred arrangement, snap fasteners are provided for removable attached of the interchangeable casing component to the chassis sub assembly or to another casing component. A snap fastener arrangement is relatively cheap to manufacture, and offers fast and easy attachment/detachment of parts by a user.

In preferred arrangements, the interchangeable casing components do not provide mechanical support for a steering control mechanism of the hand controller. For example, the steering control mechanism is not mounted on or to one or more of any interchangeable casing components. This can ensure that the casing components can be interchanged relatively easily by a user of the device, without the risk of damage or disruption to the steering control mechanism. Instead it is preferred that internal components of the hand controller such as the steering control mechanism are supported entirely by the chassis sub-assembly.

It is further contemplated that the surface texture of the removably attachable casing component(s) provided may differ between components. For example, the interchangeable casing components may include a casing component having a smooth hard surface (e.g. formed from a suitable polymer, such as polypropylene, polyamides such as nylon, polycarbonates or any other suitable polymer). The interchangeable casing components may include a casing component having a rubberized surface, or a silicone-covered surface. Some users may prefer these kinds of surface textures as they may provide for improved grip.

One or more grip features may be provided on the removably attachable casing component(s). The form of such grip features is not particularly limited, but may include or example, one or more ribs or dots provided on the removably attachable casing component. Alternatively or additionally a rubberized grip portion may be provided on the removably attachable casing component.

It is contemplated that in some cases, one or more features, such as the size, the shape, or the surface texture of the removably attachable casing component(s) provided may be selected based on one or more anthropometric or personal preference parameters of an intended user of the hand controller.

For example, prior to ordering the hand controller or kit, an intended user may submit data relating to at least one anthropometric parameter to a manufacturer of the hand controller or kit. The anthropometric parameter may include (but is not limited to) one or more of: hand length, hand breadth, grip breadth inside width diameter, and grip breadth inside length diameter.

An intended user may alternatively or additionally submit data relating to one or more personal preferences such as: curvature preference, controller size preference and surface texture preference.

One or more features of the removably attachable casing component(s) provided may then be selected based on one or more of these given anthropometric or personal preference parameters.

Accordingly, in a second aspect of Innovation D, the present invention provides a method of manufacturing a removably attachable casing component configured to provide at least part of an outer shell of a hand controller for an endoscopy system, wherein the method includes steps of:
obtaining data relating to at least one anthropometric parameter, or at least one personal preference parameter of an intended user of the endoscopy system; and
producing the removably attachable casing component, wherein one or more features of the removably attachable casing component are selected based on at least one of the obtained anthropometric parameter and/or personal preference parameters of the intended user.

The hand controller or kit according to Innovation D may be utilised in an imaging endoscope or imaging endoscopy system according to any of Innovations A, B or C, discussed above. Alternatively, it may be utilised in an otherwise conventional endoscope (which may or may not be an imaging endoscope).

### SUMMARY OF THE FIGURES

Embodiments and experiments illustrating the principles of the invention will now be discussed with reference to the accompanying figures in which:

FIG. 1 shows a schematic view of an imaging endoscopy system.

FIG. 2 is a perspective line drawing of the hand controller from a first angle.

FIG. 3 is a perspective line drawing of the same hand controller from a second angle.

FIG. 4 is a perspective line drawing of the hand controller shown in FIG. 2 and FIG. 3 with the outer casing components removed.

FIG. 5 shows a perspective line drawing of part of the steering mechanism shown in FIG. 4 (according to a first embodiment).

FIG. 6 shows a cross-section through the steering mechanism shown in FIG. 5.

FIG. 7 shows a perspective line drawing of part of a steering mechanism (according to an alternative embodiment).

FIG. 8 shows a cross-section through the steering mechanism shown in FIG. 7.

FIGS. 9a and 9b are perspective line drawings from two different angles of a rotational drive wheel.

FIG. 10 is a perspective line drawing showing an internal side of the first drive wheel body portion of the rotational drive wheel of FIGS. 9a and 9b.

FIG. 11 is a perspective line drawing showing an internal side of the second drive wheel body portion of the rotational drive wheel of FIGS. 9a and 9b.

FIG. 12 shows schematically attachment of a spacing element to a drum element of a rotational drive wheel as shown in FIGS. 5 and 6 to thereby change the apparent diameter of the drum element.

FIGS. 13(a) and (b) show side-on views of a rotational drive wheel as shown in FIGS. 5 and 6 with different diameter spacing elements disposed on the drum element.

FIGS. 14(a) and (b) show schematically two possible steering wire arrangements at a distal tip of the insertion section.

### DETAILED DESCRIPTION OF THE INVENTION

As set out above, the present inventors have realised a number of innovations which can reduce the overall cost and complexity of manufacture of an insertion section of an imaging endoscopy system, whilst providing suitable or improved performance in comparison to conventional endoscopes.

Aspects and embodiments of the present invention will now be discussed with reference to the accompanying figures. Further aspects and embodiments will be apparent to those skilled in the art. All documents mentioned in this text are incorporated herein by reference.

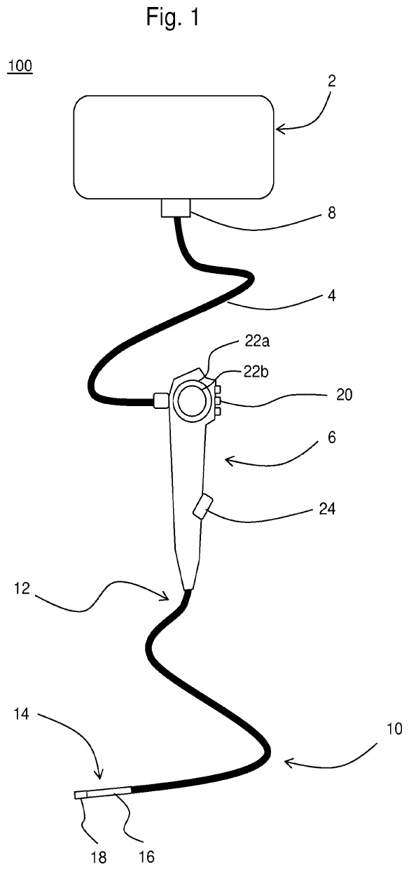

FIG. 1 shows a schematic view of an imaging endoscopy system 100 according to an embodiment of the present invention. The system includes a base unit 2 that is intended to be re-usable. Umbilical section 4 releasably connects the base unit 2 and hand controller 6 via a connector 8. Insertion section 10 has a proximal end 12 connected to the hand controller 6 and a distal end 14 for insertion into a subject. The distal end 14 has a steering section 16 and a distal tip assembly 18.

The base unit 2 is operable to provide electrical power, irrigation liquid, insufflation gas and suction via the umbilical section 4 and hand controller 6 to the insertion section 10 in a manner well known in the art (for example as described in GB2569013 B). The delivery of insufflation gas, irrigation liquid and suction are controlled using mechanical valves 20 in the hand controller 6. These valves are referred to as “trumpet” type valves in view of their similarity in appearance and operation to valve keys on a trumpet. The insufflation gas, irrigation liquid and suction are supplied from a base unit at respective suitable fixed pressures. The timing of delivery and the rate of delivery to the distal end of the endoscope is then controlled by the user operating the mechanical valves at the hand controller. The valves operate in an analogue manner (allowing different flow rates of insufflation gas and irrigation liquid, for example, based on how far they are pressed by the user).

The hand controller 6 includes a steering control mechanism for controlling bending of the steering section 16, the operation of which is described in detail below. However, in this figure, it can be seen that the steering control mechanism includes first and second user input control elements which in this embodiment are conveniently provided as first and second rotatable control wheels 22a, 22b.

The hand controller further comprises a tool insertion port 24 adapted to receive one or more surgical instruments, such as biopsy forceps.

The distal tip assembly 18 includes a light source (not shown) provided within a lighting and imaging housing portion on the distal end face of the distal tip assembly, for illumination of a region of tissue of interest. Typically this light source is an LED. At least one electrical conductor (not shown) is provided along the insertion section 10 for providing electrical power to the light source. The distal tip assembly 18 further includes an imaging chip (not shown) provided within a lighting and imaging housing portion for imaging the region of tissue of interest. The imaging chip is provided in the form of a camera, with an objective lens located to collect and direct light onto the imaging chip. An electrical conductor (not shown) is provided for conducting electrical signals from the imaging chip to the proximal end 12 of the insertion section 10. The distal tip assembly 18 has a cleaning nozzle (not shown) arranged at the distal end face, to direct irrigation liquid to clean the lighting and imaging housing portion during use.

The imaging endoscopy system 100 may be provided in kit form, including a single base unit 2, and a plurality of sealed containers which together contain a plurality of hand controllers 6, in sterile condition, a plurality of umbilical sections 4, in sterile condition, and a plurality of insertion sections 10, in sterile condition. The hand controller 6 and the umbilical section 4 and the insertion section 10 may be formed integrally in the sense that they are assembled together in the factory

When provided as a kit, for assembly of the imaging endoscopy system and readying of this for use in an endoscopic procedure, the base unit 2 is located in a suitable location for an endoscopic procedure, an operator (an endoscopist, or an assistant) opens at least one of the sealed containers, and extracts the hand controller 6, in sterile condition, the umbilical section 4, in sterile condition, and the insertion section 10, in sterile condition. The sterile umbilical section 4 is then connected to the base unit 2 via connector 8 to provide an imaging endoscopy system 100 ready for use.

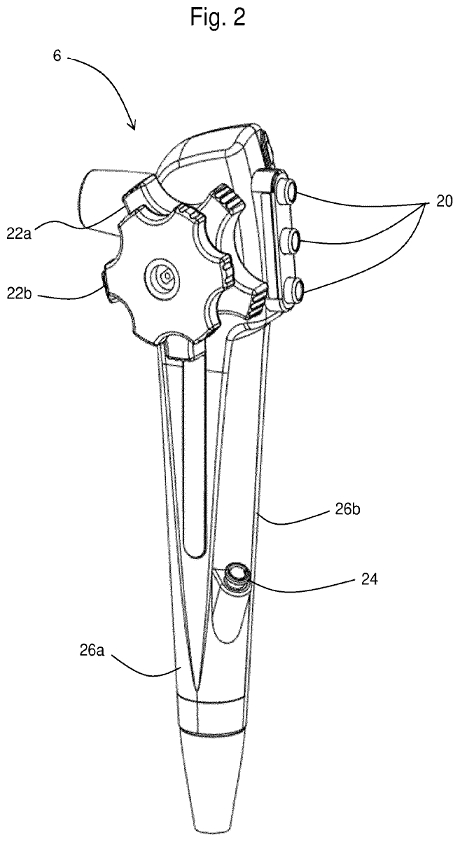

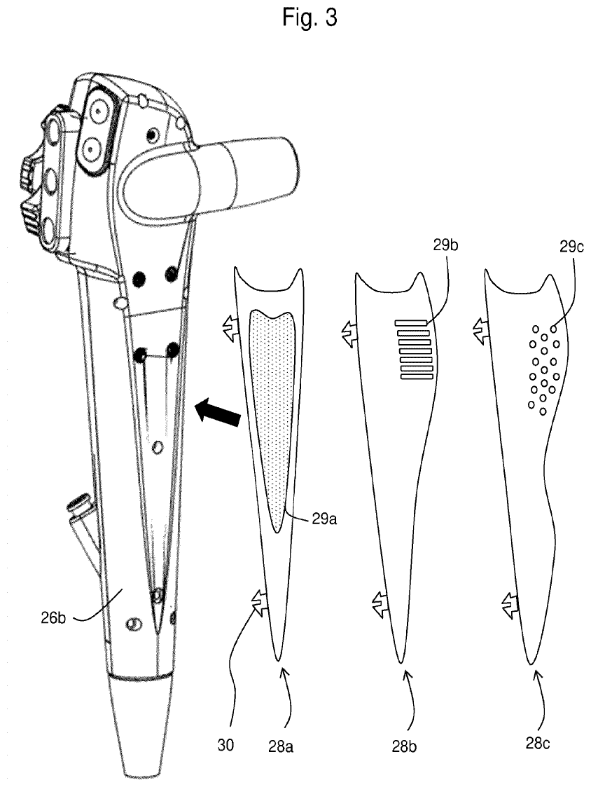

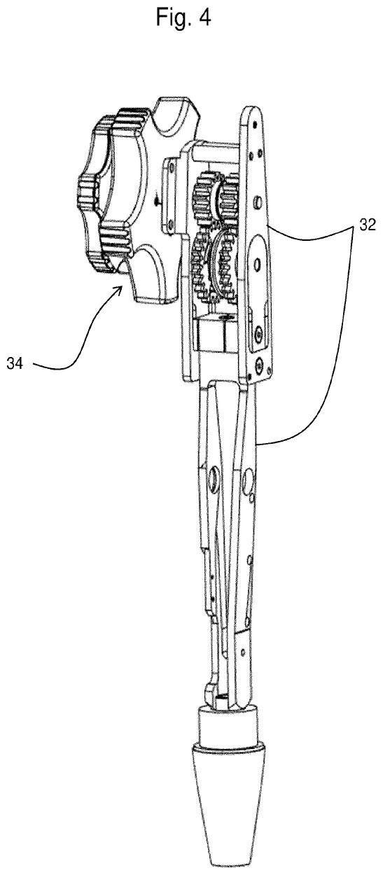

The hand controller is show in greater detail in FIG. 2, FIG. 3 and FIG. 4. FIG. 2 is a perspective line drawing of the hand controller from a first angle. FIG. 3 is a perspective line drawing of the same hand controller from a second angle.

The hand controller comprises two main injection moulded parts: a first casing component 26a and a second casing component 26b constituting the hand controller main body. These casing components are attached to one another by means of fixing, adhesive and/or (ultrasonic) welding. Furthermore, at least one is attached to the chassis sub-assembly to retain it (although this further fixing to the chassis sub-assembly is not essential—in some cases it is sufficient for the casing components to be attached to one another and surround the chassis sub-assembly without specific mechanical attachment, or be attached to the chassis sub assembly by friction-fit alone).

As shown in FIG. 3, at least one further casing component is also provided, this further casing component being removably attachable. In the embodiment shown, three interchangeable and removably attachable casing components 28a, 28b, and 28c are provided, in the form of a kit. Only one of these will be attached to the main body of the hand controller at any one time. By providing a kit in which multiple interchangeable casing components of different shapes are provided, a user of the hand controller is able to select an appropriate casing component prior to use of the hand controller, to customise the shape of the outer shell of the hand controller to meet their personal preference for controller ‘hand feel’, or to better suit the size of their hand.

The three interchangeable removably attachable casing components 28a, 28b, and 28c are each configured to be removably attachable to the second casing component 26b by means of snap fasteners 30 which are arranged for engagement with corresponding recesses provided on the second casing component 26b. The use of snap fasteners offers a convenient method of secure attachment which allows for fast and easy attachment/detachment of parts by a user when the parts are to be interchanged for a different interchangeable removably attachable casing components. Whilst in the embodiment shown, the interchangeable removable casing component is a further component that attaches to the second casing component 26b, it is also contemplated that one or both of the first casing component 26a or the second casing component 26b constituting the hand controller main body could also be an interchangeable removably attachable casing component, configured to be removably attachable to one or both of the chassis sub-assembly (described in further detail below); or to the other of the first and second casing components, to thereby provide at least part of an outer shell of the hand controller

The three interchangeable removably attachable casing components 28a, 28b, and 28c differ from one another in terms of their size and their contours: it can be seen that casing component 28a is smaller and flatter than casing components 28b and 28b. Casing component 28b provides a curved profile having a single outwardly curved section. Casing component 28c provides a curved profile having two separate outwardly curved sections. Each of the casing components, when attached, provides at least a part of the outer shell of the hand controller (it is forms part of the outermost part of the hand controller casing).

In the schematic figures shown, each of the three interchangeable removably attachable casing components 28a, 28b, and 28c comprises a different grip feature. Casing component 28a comprises a rubberized grip portion 29a. Casing component 28b comprises a plurality of ribs 29b which act as grip features. Casing component 28a comprises a plurality of dots 29c which act as grip features. Such arrangements can improve a user's grip on the hand controller. However, provision of such grip features is not essential. In some cases, a kit will be provided which includes some interchangeable removably attachable casing components which include such grip features, and some which do not include such grip features. This allows for greatest user selection of a suitable casing component to match personal preference.

FIG. 4 is a perspective line drawing of the hand controller shown in FIG. 2 and FIG. 3 with outer casing components 26a 26b removed. It can be seen that the hand controller comprises a chassis sub-assembly including a frame 32 configured to support a steering control mechanism 34 of the hand controller. By providing an arrangement in which the steering control mechanism is supported by a chassis sub-assembly, the casing components 26a, 26b, 28a, 28b, 28c are not required to provide mechanical support for a steering control mechanism of the hand controller. This can ensure that the casing components can be interchanged relatively easily, without the risk of damage or disruption to the steering control mechanism. Furthermore, as the casing components are not required to withstand significant mechanical stress, they can be manufactured more cheaply e.g. by reducing the thickness of the casing components relative to arrangements where the casing components are required with withstand significant mechanical stress.

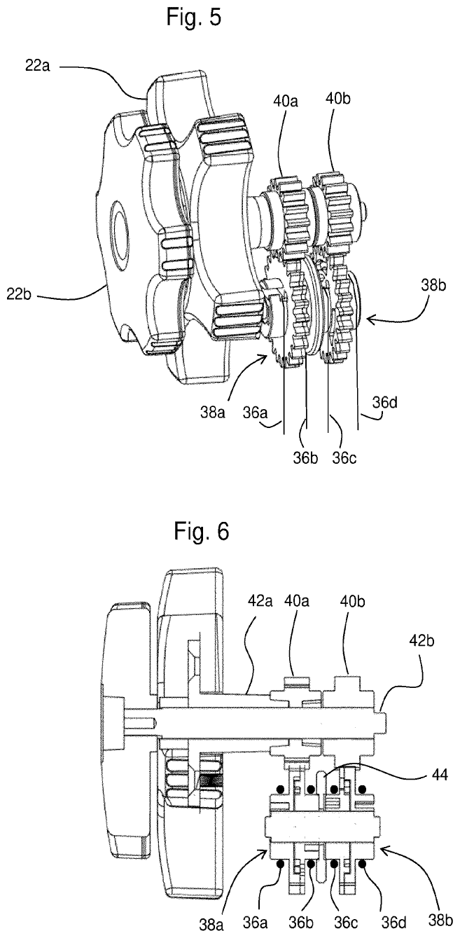

The steering control mechanism and operation thereof will now be described in greater detail, with reference to FIGS. 5 to 12.

As discussed above, the steering control mechanism includes first and second user input control elements which are conveniently provided as first and second rotatable control wheels 22a, 22b. The first and second rotatable control wheels 22a, 22b are operable by a user to apply tension to four steering wire portions 36a, 36b, 36c, 36d in order to control the bending of the steering section of the endoscope. Rotation of the first rotatable control wheel winds a first pair of steering wire portions on or off a rotational drive wheel 38a (specifically, on or off respective drum elements of the rotational drive wheel) to thereby bend the steering section in a first plane, and rotation of the second rotatable control wheel winds a second pair of steering wires on or off a rotational drive wheel 38b to thereby bend the steering section in a second plane, perpendicular to the first plane.

In the embodiment shown in FIGS. 5 and 6, each rotational drive wheel is coupled to its respective rotatable control wheel via a gearing arrangement which includes first and second gears 40a, 40b, each of these gears being arranged in a keyed configuration with respect to a respective drive shaft 42a, 42b coupled to the relevant rotatable control wheel. In this way, rotation of the first rotatable control wheel 22a causes rotation of the first drive shaft 42a, which causes rotation of the first gear 40a, which causes rotation of the first rotational drive wheel 36a. Similarly, rotation of the second rotatable control wheel 22b causes rotation of the second drive shaft 42b, which causes rotation of the second gear 40b, which causes rotation of the second rotational drive wheel 36b. A washer 44 is interposed between the first and second rotational drive wheels, to help prevent entanglement of adjacent steering wire portions 36b and 36c.

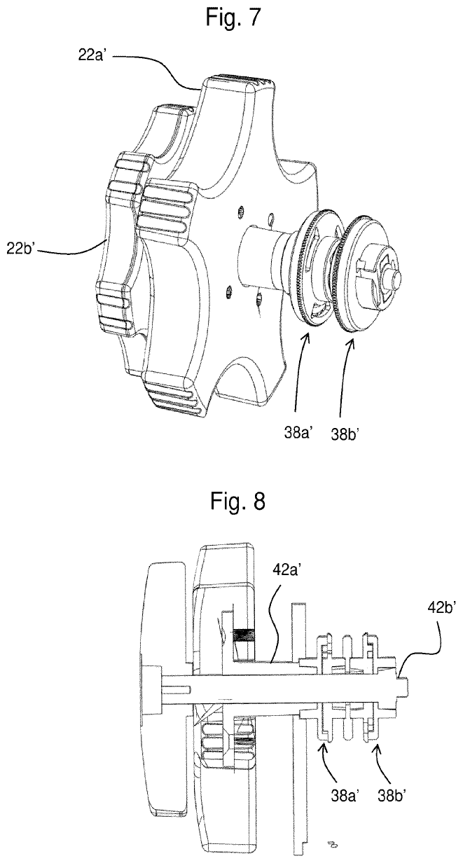

The embodiment shown in FIGS. 7 and 8 is generally similar to that shown in FIGS. 5 and 6, other than for the fact that this embodiment shows a direct drive arrangement (steering wire portions not shown in these figures). In this arrangement, each rotational drive wheel 38a′, 38b′ is coupled to its respective rotatable control wheel directly, being arranged in a keyed configuration with respect to a respective drive shaft 42a′, 42b′ coupled to the rotatable control wheel. In this way, rotation of the first rotatable control wheel 22a′ causes rotation of the first drive shaft 42a′, which causes rotation of the first rotational drive wheel 36a′. Similarly, rotation of the second rotatable control wheel 22b′ causes rotation of the second drive shaft 42b′, which causes rotation of the second rotational drive wheel 36b′.

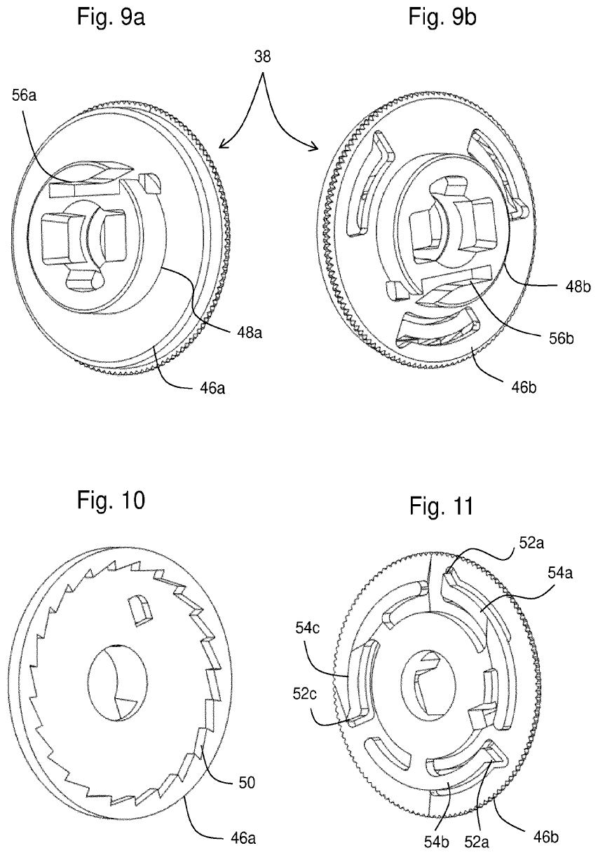

FIG. 9a, 9b, 10 and 11 show the structure of a rotational drive wheel 38 in more detail. Each rotational drive wheel comprises first and second drive wheel body portions 46a, 46b. Each of these drive wheel body portions comprises a respective drum element 48a, 48b, each drum element being configured to be coupled to a respective steering wire portion. The drum elements are integrally formed with the drive wheel body portions, each drive wheel body portion being formed as an integrally moulded polymeric component.

The rotational drive wheel comprises a ratchet mechanism configured to provide one-way relative rotation of the first drum element 48a and the second drum element 48b. In the arrangement shown, the first drive wheel body portion 46a comprises a single continuous toothed gear surface 50 which is formed as an internal toothed gear surface (i.e. it is arranged on an internal surface of the rotational drive wheel). The second drive wheel body portion 46b comprises a plurality of pawls 52a, 52b, 52c equiangularly spaced around the second drive wheel body portion, each pawl being provided as part of a flexible arm 54a, 54b, 54c integrally formed with the second drive wheel body portion. The free end of each flexible arm is arranged to engage with the teeth of the continuous toothed gear surface of the first drive wheel body portion. The pawls are spaced, when viewed along the axis of rotation of the rotational drive wheel, at positions corresponding to e.g. 120°, 240°, and 360°. In this way, the first drive wheel body portion 46a can be rotated in a first direction relative to the second drive wheel body portion 46b, to thereby cause relative rotation of the first drum element 48a and the second drum element 48b. If the body portions are rotated in the opposite direction, the pawls will engage with the teeth to prevent such rotation.

In the arrangement shown, the first drum element 48a and the second drum element 48b each comprise respective wire-receiving recesses 56a, 56b in which ends of the first steering wire and the second steering wire are respectively affixed. Whilst not shown in these figures, a ferrule is located in each of these wire-receiving recesses. During manufacture, an end of the relevant steering wire is inserted into the ferrule, and the ferrule then clamped to affix the end of the steering wire in the wire-receiving recess. The steering wire portions on each side of the rotational drive wheel are then counter-wound onto their respective drum elements (i.e. wound in opposite directions).

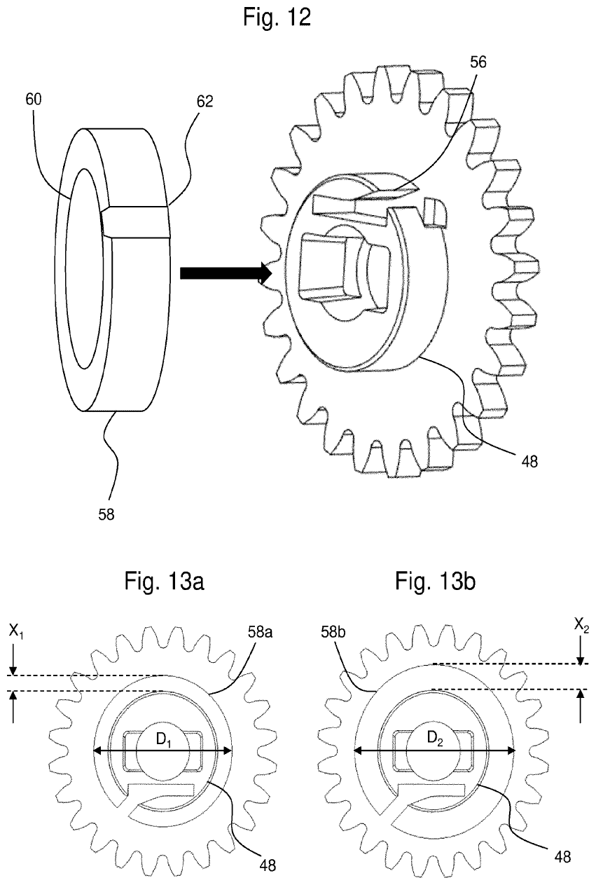

As shown in FIG. 12 and FIG. 13, in some arrangements, one or more spacing elements 58 may be provided which are configured to be disposed on the first and/or second drum elements to thereby change the apparent diameter of the first and/or second drum elements. The spacing element shown in this figure is in the form of a ring member having a central aperture 60 suitably sized to fit around the drum element 48 and be held in a friction-fit arrangement. The spacing element further comprises a cut-out portion 62 which acts as a wire-receiving slot arranged to align with the wire-receiving recess 56 of the drum element. A plurality of such spacing elements may be provided, as part of a kit—for example, the spacing elements 58a and 58b shown in FIG. 13a and FIG. 13b may be provided as part of the same kit. These spacing elements are ring members having different thicknesses X₁ and X₂, such that when fitted onto drum element 48, the apparent diameter of the drum element is defined as D₁ or D₂ respectively (D₂ being larger than D₁). The spacing elements in the kit may differ in size to thereby provide for a range of different effective diameters of the drum element.

During manufacture, the first and second drive wheel body portions can be rotated relative to one another in the direction provided by the ratchet mechanism to thereby wind a selected amount of wire onto the drum elements. This may be performed e.g. to set a predetermined tension in one or both of the wires forming part of the steering control mechanism.

After manufacture, and during use of the steering control mechanism for steering of the endoscope, rotation of the entire rotational drive wheel in a first direction will cause a first steering wire portion will be wound onto the wheel, whilst a second steering wire portion is wound off the wheel, with the result that the increasing length change of one is accommodated by a corresponding shortened length change in the other, to thereby direct bending of the steering section in a first plane. Where a spacing element is present, this will changing the steering response of the steering control mechanism: by providing a larger apparent diameter of the drum elements, a set user input may result in a larger steering response (i.e. greater deflection of the steering section of the endoscope). By providing a smaller apparent diameter of the drum elements, a set user input may result in a smaller steering response (i.e. smaller deflection of the steering section of the endoscope).

Whilst not shown in the present figures, each of the steering wires portions are provided as part of a Bowden cable, i.e. each wire portion comprises an outer constant length cable sheath defining an axis of constrained movement for each respective steering wire portion. The outer constant length cable sheaths are connected proximally to the hand controller and distally at the proximal link of the steering section in four places (again, not shown). These four connection locations are equiangularly spaced around the insertion section of the endoscope, when viewed along the axis of the insertion section of the endoscope, for example at positions corresponding to 90°, 180°, 270° and 0/360°. The steering wire portions which pass through the constant length cable sheaths, pass through the steering section and are connected to the distal end of the steering section.

As described above, proximally the steering wire portions have ends which are coupled to the drum elements. For each axis of bending there is a respective drive wheel, which, when turned either clockwise or counter clockwise pulls one of the steering wire portions and causes movement of the corresponding steering wire at 180° radially to it at the distal end of the steering section. This has the effect of bending the steering section until rotation of the drive wheel is stopped. Accordingly, for the 90° and 270° steering wires, these form a first pair in the sense that a length change of one is accommodated by a corresponding length change in the other and they direct bending of the steering section in a first plane. For the 180° and 0/360° steering wires, these form a second pair in the sense that a length change of one is accommodated by a corresponding length change in the other and they direct bending of the steering section in a second plane, perpendicular to the first plane.

The four steering wire portions are provided by two continuous steering wires having first and second ends coupled to a user control assembly at the hand controller, and an intermediate portion fixed at the distal tip of the insertion section, each of the two continuous steering wires thereby defining two steering wire portions extending from the hand controller to the distal tip, for a total of four steering wire portions as discussed above.

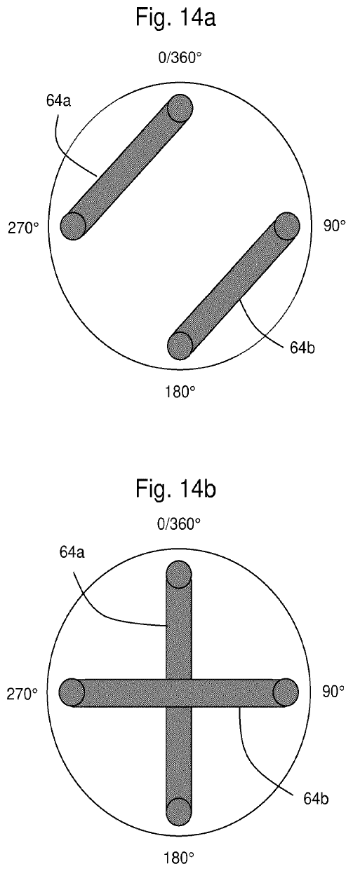

FIGS. 14(a) and (b) show schematically two possible steering wire arrangements for these two continuous steering wires, as viewed from a distal tip of the insertion section. In FIG. 14a, each of the pairs of opposed steering wire portions configured to direct bending in a particular plane (i.e. the 90° & 270° steering wire portion pair, and the 180° & 0/360° steering wire portion pair) includes one portion constituting part of a first continuous steering wire 64a, and one portion constituting part of a second continuous steering wire 64b. The first and second continuous steering wires each have a first end attached to the first rotational drive wheel and a second end attached to the second rotational drive wheel—for example, in the arrangement shown in FIG. 5, steering wire portions 38a and 38c are both portions of the same continuous steering wire, and steering wire portions 36b and 36d are both portions of another continuous steering wire. An intermediate portion of each continuous steering wire is fixed at the distal tip of the insertion section (fixing not shown in this schematic figure) to thereby define the respective steering wire portions which extend along the insertion section. Conveniently, the intermediate portion of the continuous steering wires are threaded though a hole formed in the distal tip of the steering section before a mid loop knot such as a ‘FIG. 8’ knot is tied, with the knot then acting to prevent the wire being pulled through said hole, thereby fixing the intermediate portion of the continuous steering wire at the distal tip. This has been found to provide a simple to manufacture, low-cost, yet effective construction.

FIG. 14b shows an alternative arrangement, in which each pair of opposed steering wire portions are part of the same continuous steering wire, i.e. the 180° & 0/360° steering wire portion pair are both part of the first continuous steering wire 64a and the 90° & 270° steering wire portion pair are both part of the second continuous steering wire 64b. Similarly to the arrangement shown in FIG. 14a, an intermediate portion of each continuous steering wire is fixed at the distal tip of the insertion section (fixing not shown in this schematic figure) to thereby define the respective steering wire portions which extend along the insertion section.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

1. An imaging endoscopy system comprising:
a hand controller;
an insertion section having a proximal end connected to the hand controller and a distal end for insertion into a subject, the distal end having a steering section and a distal tip;
a steering control mechanism comprising a first continuous steering wire, said first continuous steering wire having first and second ends coupled to a user control assembly at the hand controller, and an intermediate portion fixed at the distal tip of the insertion section, thereby defining two steering wire portions extending from the hand controller to the distal tip, said steering wire portions capable of being tensioned to cause bending of the steering section in response to operation of the user control assembly of the hand controller.

2. The imaging endoscopy system according to claim 1, further comprising a second continuous steering wire, said second continuous steering wire having first and second ends coupled to the user control assembly at the hand controller, and an intermediate portion fixed at the distal tip of the insertion section, thereby defining two further steering wire portions extending from the hand controller to the distal tip, said steering wire portions capable of being tensioned to cause bending of the steering section in response to operation of the user control assembly of the hand controller.

3. The imaging endoscopy system according to claim 2 wherein the four steering wire portions are substantially equiangularly spaced around the insertion section when viewed in cross section.

4. The imaging endoscopy system according to claim 2 wherein a first pair of opposed steering wire portions of the four steering wire portions are configured to cooperate to steer the distal tip in a first predetermined plane, and wherein a second pair of opposed steering wire portions of the four steering wire portions are configured to cooperate to steer the distal tip in a second predetermined plane.

5. The imaging endoscopy system according to claim 4 wherein each pair of opposed steering wire portions are part of the same continuous steering wire.

6. The imaging endoscopy system according to claim 4 wherein each pair of opposed steering wire portions includes one portion constituting part of the first continuous steering wire, and one portion constituting part of the second continuous steering wire.

7. The imaging endoscopy system according to claim 1 wherein the intermediate portion of the first and/or second continuous wire portions is fixed at the distal tip via one or more of a clamping member, a knot, or an adhesive.

8. The imaging endoscopy system according to claim 7 wherein the intermediate portion of the first and/or second continuous wire portions is threaded through a hole formed in the distal tip of the steering section, fixed via a knot arranged distally of said hole.

9. The imaging endoscopy system according to claim 7 wherein the intermediate portion of the first and/or second continuous wire portions is fixed at the distal tip by a ‘FIG. 8’ knot.

10. The imaging endoscopy system according to claim 1 wherein the user control assembly of the steering control mechanism includes one or more user input control elements selected from one or more rotatable control wheels, one or more switches, one or more levers or one or more buttons.

11. The imaging endoscopy system according to claim 2 wherein the steering control mechanism comprises first and second rotational drive wheels, wherein the first and second continuous steering wires each have a first end coupled to the first rotational drive wheel and a second end coupled to the second rotational drive wheel.

12. The imaging endoscopy system according to claim 11 wherein each of the first and second rotational drive wheels comprise first and second drive wheel body portions having respective drum elements, and the ends of the first and second steering wires are coupled to the rotational drive wheels by being attached to the drum elements of the drive wheels.

13. The imaging endoscopy system according to claim 12 wherein the rotational drive wheels are configured to provide one-way relative rotation of their respective first and second drum elements.

14. The imaging endoscopy system according to claim 1 wherein each steering wire portion comprises a respective outer constant length cable sheath defining an axis of constrained movement for its respective steering wire portion.
